(19) United States
(12) Patent Application Publication
(10) Pub. No.: US 2016/0287658 A1
(43) Pub. Date: Oct. 6, 2016

(54) COMPOSITION FOR MOISTURIZING SKIN

(71) Applicant: AMOREPACIFIC CORPORATION, Seoul (KR)

(72) Inventors: Eui Dong SON, Yongin-si (KR); Hyun Seo KANG, Yongin-si (KR); Hyeon Chung KIM, Yongin-si (KR); Yoon Kyung KIM, Yongin-si (KR); Eun Gyung CHO, Yongin-si (KR)

(21) Appl. No.: 15/080,942

(22) Filed: Mar. 25, 2016

(30) Foreign Application Priority Data
Mar. 31, 2015 (KR) 10-2015-0045066

(51) Int. Cl. A61K 36/899; A61K 8/44; A61K 31/205; A61Q 19/00; A61K 8/97

(52) U.S. Cl. CPC A61K 36/899; A61Q 19/007; A61K 8/97; A61K 31/205; A61K 8/44; A23L 1/3002; A23V 2002/00

(57) ABSTRACT

In an aspect, the present disclosure provides a composition for moisturizing skin, which contains a bamboo sap and a betaine as active ingredients. Specifically, the composition exhibits skin moisturizing effect by regulating natural moisturizing factors (NMFs) and is useful as a cosmetic composition, a pharmaceutical composition or a health food composition effective in moisturizing skin.

### CROSS-REFERENCE TO RELATED APPLICATION

This application claims priority to Korean Patent Application No. 10-2015-0045066, filed on Mar. 31, 2015, and all the benefits accruing therefrom under 35 U.S.C. §119, the contents of which in its entirety are herein incorporated by reference.

### BACKGROUND

1. Field

The present disclosure relates to a composition effective in moisturizing skin.

2. Description of the Related Art

Skin is an organ which is located at the outermost part of the human body and protects the body. It prevents loss of water and electrolytes from the body and provides protection from invasion of foreign materials. The skin is composed of the epidermis, the dermis and the subcutaneous fat layer. The epidermis is composed of corneocytes. The stratum corneum, the outermost layer of the epidermis, plays a very important role in transport of materials.

The epidermis is composed of the stratum basale, the stratum spinosum, the stratum granulosum and the stratum corneum. The stratum corneum is composed of corneocytes and intercellular lipids. The stratum corneum prevents loss of water and can function appropriately when it contains water in an appropriate amount. The barrier of the stratum corneum is composed of ceramides, fatty acids and cholesterols. It prevents invasion of foreign materials and prevents transepidermal water loss (TEWL). Also, natural moisturizing factors (NMFs) present in the stratum corneum are known to play an important role in moisturizing of skin. Of the water contained in the epidermis, about 70% is present in the stratum basale and the stratum spinosum, 30-50% is present in the stratum granulosum and 15-30% is present in the stratum. That is to say, the epidermis exhibits a skin water gradient with the water content decreasing toward the upper layer.

Because the skin is located at the outermost part of the human body, it is chronically exposed to air pollution, dry environment, UV, wind, chemicals, etc. These conditions render the skin under continued osmotic stress and accelerate skin dryness. Recently, as the number of people with dry skin increases, demand on moisturizers is also increasing.

### SUMMARY

The present disclosure is directed to providing a composition for moisturizing skin, which is effective in moisturizing skin.

The present disclosure is also directed to providing a composition which regulates natural moisturizing factors (NMFs).

The present disclosure is also directed to providing a composition which regulates natural moisturizing factors by increasing the expression of the filaggrin gene, increasing the expression of the bleomycin hydrolase gene or both.

The present disclosure is also directed to providing a cosmetic composition, a pharmaceutical composition or a health food composition, which is effective in moisturizing skin.

The present disclosure is also directed to providing a kit for moisturizing skin, which includes: a first composition containing a bamboo sap; and a second composition containing a betaine.

In an aspect, the present disclosure provides a composition for moisturizing skin, which contains a bamboo sap and a betaine as active ingredients.

In another aspect, the present disclosure provides a composition for regulating natural moisturizing factors (NMFs), which contains a bamboo sap and a betaine as active ingredients. Specifically, the active ingredients regulate natural moisturizing factors by increasing the expression of the filaggrin gene, increasing the expression of the bleomycin hydrolase gene or both.

In another aspect, the present disclosure provides a composition wherein the bamboo sap is the sap of one or more of Phyllostachys bambusoides, Phyllostachys nigra var. henonis and Phyllostachys pubescens.

In another aspect, the present disclosure provides a composition wherein the betaine is glycine betaine.

In another aspect, the present disclosure provides a composition wherein a weight ratio of the bamboo sap and the betaine as the active ingredients is 1:0.1 to 1:10.

In another aspect, the present disclosure provides a composition wherein the bamboo sap and the betaine are respectively contained in an amount of 0.001-80 wt % based on the total weight of the composition.

In another aspect, the present disclosure provides a composition wherein the composition is a cosmetic composition, a pharmaceutical composition or a health food composition. Specifically, the pharmaceutical composition is a composition which is used to treat or prevent one or more skin dryness disease selected from atopy, xeroderma, xerotic eczema, psoriasis and xeroderma pigmentosum.

In another aspect, the present disclosure provides a kit for moisturizing skin, which includes: a first composition containing a bamboo sap; and a second composition containing a betaine.

The composition according to an aspect of the present disclosure is remarkably effective in moisturizing skin.

The composition according to an aspect of the present disclosure is effective in regulating natural moisturizing factors (NMFs).

The composition according to an aspect of the present disclosure is effective in increasing the expression of the filaggrin gene.

The composition according to an aspect of the present disclosure is effective in increasing the expression of the bleomycin hydrolase gene.

### BRIEF DESCRIPTION OF THE DRAWINGS

FIG. 1 shows a result of comparing the expression level of the filaggrin and bleomycin hydrolase (BH) genes in keratinocytes after treating with a bamboo sap and a betaine at the same time.

FIG. 2 shows a result of comparing the amount of natural moisturizing factors (NMFs) in keratinocytes after treating with a bamboo sap and a betaine at the same time.

FIG. 3 shows a result of measuring water content after applying a composition containing a bamboo sap and a betaine to skin.

### DETAILED DESCRIPTION

The main function of the stratum corneum is to protect skin by preventing evaporation of water from the skin or invasion of harmful materials from outside. The stratum corneum contains approximately 30% of water based on its dry weight, 20% in corneocytes and 10% in intercellular lipids. The water in corneocytes is present as bound to natural moisturizing factors (NMFs) formed from degradation of filaggrin. In atopy and dry skin, bleomycin hydrolase, which is the enzyme necessary to convert filaggrin to NMFs, is decreased.

In the present disclosure, a bamboo sap and a betaine are used as moisturizing ingredients. When the bamboo sap and the betaine are used together according to the present disclosure, the NMFs in the stratum corneum are increased remarkably and, as a result, the water content in the stratum corneum is increased remarkably as compared to when they are used alone.

That is to say, according to the present disclosure, a remarkable effect of moisturizing skin is achieved due to a combination of the filaggrin production promoting ability of the bamboo sap and the bleomycin hydrolase activating ability of the betaine.

In an aspect, the present disclosure relates to a composition for moisturizing skin, which contains a bamboo sap and a betaine as active ingredients.

In an aspect, the present disclosure provides a method for moisturizing the skin of a subject, which includes a step of administering an effective amount of a bamboo sap and a betaine to a subject in need thereof.

In an aspect, the present disclosure relates to a composition for regulating natural moisturizing factors (NMFs), which contains a bamboo sap and a betaine as active ingredients.

Specifically, it relates to a composition for regulating natural moisturizing factors (NMFs), wherein the active ingredients regulate the natural moisturizing factor by increasing the expression of the filaggrin gene, increasing the expression of the bleomycin hydrolase gene or both.

In general, a bamboo sap is produced as a byproduct while bamboo is cut periodically to help its growth. It usually contains proteins (0.08-0.7%), calcium (422 mg/L), sugars (1.6-2.7 g/L), amino acids (7.6-30 mg/L), etc., although the compositing varies depending on particular region, species, time of harvesting, etc., and is known to have a pH of about 4.5.

In an aspect of the present disclosure, the bamboo sap may be obtained specifically from Phyllostachys bambusoides, Phyllostachys nigra var. henonis and Phyllostachys pubescens, although not being limited thereto. As is well known, it may be harvested by making a cut right above the first or second joint (10-20 cm in height) from the root and attaching a container thereto and then filtered through filter cloth. The filtrate may be sterilized for use as a cosmetic composition. Alternatively, it may be used as it is or after being prepared into powder. Specifically, the bamboo sap may be harvested around May and June.

Immediately after harvesting, the bamboo sap may be pretreated by filtering through a filtration membrane with a pore size of 0.001-0.45 μm, specifically 0.2 μm, and adding 7.0 wt % or more, specifically 7.0-50 wt %, of anhydrous ethanol to the filtrate. When the pore size of the filtration membrane is smaller than 0.001 μm, a long time is required for the filtration. And, when it exceeds 0.45 μm, filtration efficiency decreases.

That is to say, after removing microbes, microorganisms and high-molecular-weight proteins that are likely to precipitate or be denatured during long-term storage of the bamboo sap by filtering through a filtration membrane, anhydrous ethanol may be added to inhibit enzymatic activity and thereby prevent discoloration or change in odor.

The pretreated a bamboo sap is stored under nitrogen atmosphere until use to prevent intrusion of microbes or microorganisms from outside.

In the present disclosure, the bamboo sap may be used as an active ingredient of a cosmetic or pharmaceutical composition for moisturizing skin as it is or after being pretreated to prevent discoloration or change in odor during storage before use.

The bamboo sap pretreated as described above may be used as an active ingredient of a composition for moisturizing skin.

In an aspect of the present disclosure, the betaine refers a salt compound having a quaternary ammonium ion as a cation and an acid anion, particularly a carboxylate anion, in the molecule. However, in solution or bound state, it actually exists as a zwitterion and is thought of as a dehydration product of a quaternary ammonium hydroxide compound having a carboxylate group. In the present disclosure, a hydrate is also called a betaine. In general, a betaine is a crystal with a high melting or decomposition point and is water-soluble due to the polar groups. It dissolves well in water and the pH of an aqueous solution (1→20) is 5.0-7.0. It is contained in squid, octopus, etc. and is used as a condiment in processed marine products. The betaine is widely distributed in animals and plants. It is present in the muscle of fish in an amount of 0.1% or less. It is more abundant in the muscle of invertebrate animals such as squid, octopus, shrimp, etc. than in fish and is also contained in beet, bean, radish, Chinese matrimony vine, etc. The betaine has anti-fatty liver activity, blood pressure lowering activity, blood sugar lowering activity, eyesight recovering effect, detoxification activity, cell reproducing activity, etc. In particular, it promotes insulin by stimulating Langerhans cells in the pancreas, exhibits an anti-fatty liver activity of treating alcohol-induced fatty liver and protects the kidneys by producing carnitine. In addition, it prevents cardiovascular diseases such as atherosclerosis by inhibiting homocysteine which causes cardiovascular diseases at elevated level in blood.

In an aspect, the betaine used in the present disclosure may be glycine betaine, histidine betaine, proline betaine, tyrosine betaine, phenylalanine betaine, tryptophan betaine, leucine betaine, isoleucine betaine, valine betaine, alanine betaine, betaine glutamate, betaine aspartate, lysine betaine, betaine arginine, etc. depending on the amino acid involved.

In an aspect, the betaine used in the present disclosure may be an alkyl betaine, an alkyl amidobetaine, an alkyl sulfobetaine, an imidazolinium betaine, etc. Specifically, an alkyl N-betaine, an alkyl N-sulfobetaine, an acyl N-betaine, an alkyl N-substituted aminopropionic acid, an alkyl imidazolinium betaine, etc. may be used.

More specifically, glycine betaine, or trimethylglycine may be used.

In an aspect of the present disclosure, a derivative of the betaine may be used. For example, an organic acid salt, an inorganic acid, a base salt, etc. may be used. Specifically, betaine hydrochloride, betaine hydrobromide, betaine hydroiodide, betaine chloroaurate, betaine hydrazide hydrochloride, etc. may be used.

In an aspect of the present disclosure, a weight ratio of the bamboo sap and the betaine in the mixture is 1:0.1 to 1:10.

In an aspect, the weight ratio of the bamboo sap and the betaine may be 1:0.1 or greater, 1:0.2 or greater, 1:0.3 or greater, 1:0.4 or greater, 1:0.5 or greater, 1:0.6 or greater, 1:0.7 or greater, 1:0.8 or greater, 1:0.85 or greater, 1:0.90 or greater, 1:0.92 or greater, 1:0.94 or greater, 1:0.96 or greater, 1:0.98 or greater or 1:1 or greater.

In an aspect, the weight ratio of the bamboo sap and the betaine may be 1:1 or smaller, 1:9 or smaller, 1:8 or smaller, 1:7 or smaller, 1:6 or smaller, 1:5 or smaller, 1:4 or smaller, 1:3 or smaller, 1:2 or smaller, 1:1.8 or smaller, 1:1.6 or smaller, 1:1.4 or smaller, 1:1.2 or smaller or 1:1.0 or smaller.

When the weight ratio of the bamboo sap and the betaine is within the above-described range, a remarkable effect of moisturizing skin can be achieved as compared to when the bamboo sap or the betaine is used alone. In particular, natural moisturizing factors (NMFs) are increased remarkably.

The composition according to the present disclosure may be used in various fields without particular limitation. For example, it may be formulated into a composition for external application to skin, specifically a cosmetic or pharmaceutical composition.

The composition according to an aspect of the present disclosure may contain 0.001 wt %-80 wt %, specifically 0.005-60 wt %, more specifically 0.01-30 wt %, of a mixture of the bamboo sap and the betaine based on the total weight of the composition. When the mixture is contained within the above-described range, the effect desired by the present disclosure can be achieved appropriately. In addition, both the stability and safety of the composition can be satisfied and it may be favorable in terms of cost effectiveness. Specifically, when the amount of the mixture of the bamboo sap and the betaine is less than 0.001 wt %, a sufficient skin moisturizing effect may not be achieved. And, when it exceeds 80 wt %, the stability and safety of the composition may be unsatisfactory.

In an aspect, the present disclosure provides a cosmetic composition, which contains mixture of the bamboo sap and the betaine as an active ingredient. The cosmetic composition may enhance skin moisturizing effect and it may be a functional cosmetic composition.

The cosmetic composition according to the present disclosure may be formulated into a solution, suspension or emulsion in an oily or aqueous medium or may be formulated into a dry powder which is dissolved in sterilized water for use. A water-in-oil emulsion may be prepared by using a vegetable oil such as olive oil or a mineral oil such as liquid paraffin as an oil phase and emulsifying the active ingredient using a natural phospholipid such as soybean lecithin, an anhydrous hexitol such as sorbitan monooleate, a fatty acid-derived ester or a condensation product of an anhydrous hexitol- and fatty acid-derived ester with ethylene oxide such as polyoxyethylene sorbitol monooleate as an emulsifying agent.

In an exemplary embodiment, the cosmetic composition according to the present disclosure may contain 0.001-80 wt % of the mixture of the bamboo sap and the betaine based on the total weight of the composition. When the content of the mixture is less than 0.001 wt %, skin moisturizing effect may be insignificant. And, when it exceeds 80 wt %, the efficiency of adding the mixture may decrease and the stability of the composition may be unsatisfactory.

The cosmetic composition may further contain, in addition to the active ingredient, other ingredients commonly used in the art depending on the particular formulation or purpose of use. These ingredients may be adequately selected by those skilled in the art without difficulty and a synergic effect may be achieved in that case.

The cosmetic composition may be used for the purpose of moisturizing skin. The formulation of the cosmetic composition is not particularly limited. For example, it may be in the form of a softening lotion, a nourishing lotion, a massage cream, a nourishing cream, a pack, a gel or a skin adhesive type cosmetic. Also, it may be in the form of formulation for transdermal administration such as a lotion, an ointment, a gel, a cream, a patch or a spray. Those skilled in the art may add the other ingredients considering the type or purpose of the cosmetic composition.

In an aspect, the present disclosure provides a pharmaceutical composition, which contains the mixture of the bamboo sap and the betaine as an active ingredient. The pharmaceutical composition is effective for various skin dryness diseases. Specifically, the pharmaceutical composition may be used for one or more of atopy, xeroderma, xerotic eczema, psoriasis and xeroderma pigmentosum. However, the therapeutic use of the present disclosure is not limited thereto and it will be obvious to those skilled in the art that it can be used for any skin disease associated with skin dryness.

The pharmaceutical composition according to an aspect of the present disclosure may be orally or parenterally, e.g., rectally, topically, transdermally, intravenously, intramuscularly, intraperitoneally or subcutaneously.

Formulations for oral administration may include a tablet, a pill, a soft or hard capsule, a granule, a powder, a fine granule, a liquid, an emulsion or a pellet, although not being limited thereto. These formulations may further contain, in addition to the active ingredient, a diluent (e.g., lactose, dextrose, sucrose, mannitol, sorbitol, cellulose or glycine), a lubricant (e.g., silica, talc, stearic acid or polyethylene glycol) or a binder (e.g., magnesium aluminum silicate, starch paste, gelatin, tragacanth, methyl cellulose, sodium carboxymethyl cellulose or polyvinylpyrrolidone). If necessary, other pharmaceutical additives such as a disintegrant, an absorbent, a colorant, a flavor, a sweetener, etc. may be further contained. The tablet may be prepared by commonly employed mixing, granulation or coating methods.

Formulations for parenteral administration may include an injection, a drip, a lotion, an ointment, a gel, a cream, a suspension, an emulsion, a suppository, a patch or a spray, although not being limited thereto.

The administration dosage of the active ingredient of the pharmaceutical composition according to an aspect of the present disclosure may vary depending on the age, sex and body weight of a subject to be treated, a pathological condition and severity thereof, administration route and discretion of a diagnoser. Determination of the administration dosage considering these factors is within the level of those skilled in the art. A daily administration dosage may be, for example, 0.1-500 mg/kg/day, more specifically 5-100 mg/kg/day, although not being limited thereto.

In an aspect, the present disclosure provides a health food composition, which contains the mixture of the bamboo sap and the betaine as an active ingredient.

The active ingredients of the present disclosure, i.e., the bamboo sap and the betaine, may be added to a health food for the purpose of moisturizing skin. When used as a food additive, the bamboo sap and the betaine may be added as they are or as being mixed with other foods or food ingredients according to common methods. The content of the active ingredients may be determined adequately depending on the purpose of use (e.g., prevention, health promotion or treatment). In general, the betaine as the active ingredient in the present disclosure is added in an amount of 80 wt % or less, specifically 50 wt % or less, when preparing a food or drink. However, in case of long-term intake for the purpose of health or hygiene improvement, the addition amount may be below the above-described range. In addition, the addition amount of the active ingredient may be above the above-described range if there is no problem in terms of safety.

The type of the food is not particularly limited. Examples of the food to which the active ingredient can be added include meat, sausage, bread, chocolate, candy, snack, confectionery, pizza, instant noodle, other noodles, gum, dairy products including ice cream, soup, beverage, tea, drink, alcoholic drink, vitamin complex, etc. In addition, any health food in common sense is included.

A health drink composition of the present disclosure may further contain various flavors or natural carbohydrates commonly used in drinks. The natural carbohydrate may be a monosaccharide such as glucose and fructose, a disaccharide such as maltose and sucrose, a polysaccharide such as dextrin and cyclodextrin and a sugar alcohol such as xylitol, sorbitol, erythritol, etc. As a sweetener, a natural sweetener such as thaumatin and stevia extract or a synthetic sweetener such as saccharin and aspartame may be used.

In addition, the health food of the present disclosure may contain various nutrients, vitamins, electrolytes, flavors, colorants, pectic acid and its salts, alginic acid and its salts, organic acids, protective colloidal thickener, pH control agents, stabilizers, antiseptics, glycerin, alcohols, carbonating agent used in carbonated drinks, etc. In addition, the health food of the present disclosure may further contain a pulp for preparing a natural fruit juice, a fruit juice drink or a vegetable juice. These ingredients may be used either independently or in combination. The content of these additives is of no great importance. Usually, they are used in an amount of 0.1-30 parts by weight based on 100 parts by weight of the betaine as the active ingredient of the present disclosure.

Determination of the administration dosage of the active ingredient is within the level of those skilled in the art. A daily administration dosage may be, for example, 0.1-5000 mg/kg/day, more specifically 50-500 mg/kg/day, although not being limited thereto. The administration dosage may vary depending on various factors including the age of a subject, health condition, presence of complication(s), etc.

In an aspect, the present disclosure provides a kit for moisturizing skin, which includes: a first composition containing a bamboo sap; and a second composition containing a betaine. Specifically, the kit may contain the bamboo sap and the betaine in an amount of 0.001-80 wt % based on the total weight of the first composition and the second composition, respectively. The kit may include an instruction and the instruction may dictate that the first composition containing the bamboo sap and the second composition containing the betaine are administered at a weight ratio of 1:0.1-1:10. The first composition containing the bamboo sap and the second composition containing the betaine may be administered to a subject either simultaneously or sequentially.

Hereinafter, the present disclosure will be described in detail through examples and test examples. However, the following examples and test examples are for illustrative purposes only and it will be apparent to those of ordinary skill in the art that the scope of the present disclosure is not limited by them.

### EXAMPLE 1

### Bamboo Sap

A bamboo sap was harvested by making a cut right above the first or second joint (10-20 cm in height) from the root and attaching a container thereto. The bamboo sap was filtered through 200-mesh filter cloth. 100 g of the obtained bamboo sap was prepared into 0.21 g of powder by concentrating under reduced pressure by a common method. The powder was used in experiments using cells and the sap was used in Preparation Examples.

### EXAMPLE 2

### Betaine

As a betaine, trimethylglycine (Danisco, CAS No. 107-43-7) in white powder form was used as glycine betaine.

### EXAMPLE 3

### Mixture of Bamboo Sap and Betaine

The bamboo sap and the trimethylglycine were mixed at a weight ratio of 1:1.

### PREPARATION EXAMPLES 1-3 AND COMPARATIVE EXAMPLE 1

A composition of Preparation Example 1 was prepared with the compositional ratio (wt %) of Table 1 using the bamboo sap of Example 1.

A composition of Preparation Example 2 was prepared with the compositional ratio of Table 1 using the betaine of Example 2.

A composition of Preparation Example 3 was prepared with the compositional ratio of Table 1 using the 1:1 mixture of the bamboo sap and the betaine of Example 3.

A composition of Comparative Example 1 was prepared with the compositional ratio of Table 1 without adding an active ingredient, as a control group.

TABLE 1

| | Comparative | Preparation | Preparation | Preparation |
|---|---|---|---|---|
| Ingredients | Example 1 | Example 1 | Example 2 | Example 3 |
| Purified water | balance | balance | balance | balance |
| Bamboo sap (0.1%) | — | 0.1 | — | 0.05 |
| Betaine (0.1%) | — | — | 0.1 | 0.05 |
| Hydrogenated vegetable oil | 1.5 | 1.5 | 1.5 | 1.5 |
| Stearic acid | 0.6 | 0.6 | 0.6 | 0.6 |
| Glyceryl stearate | 1 | 1 | 1 | 1 |
| Stearyl alcohol | 2.5 | 2.5 | 2.5 | 2.5 |
| Polyglycerol-10 pentastearate, | 1 | 1 | 1 | 1 |
| benhenyl alcohol & sodium | | | | |
| stearoyl lactylate | | | | |
| Arachidyl behenyl alcohol & | 1 | 1 | 1 | 1 |
| arachidyl glucoside | | | | |
| Cetearyl alcohol& cetearyl | 2 | 2 | 2 | 2 |
| glucoside | | | | |
| PEG-100 stearate, glycerol oleate | 1.5 | 1.5 | 1.5 | 1.5 |
| & propylene glycol | | | | |
| Caprylic/capric triglyceride | 11 | 11 | 11 | 11 |
| Cyclomethicone | 6 | 6 | 6 | 0.6 |
| Antiseptic & fragrance | adequate | adequate | adequate | adequate |
| Triethanolamine | 0.1 | 0.1 | 0.1 | 0.1 |

### TEST EXAMPLE 1

### Increased Expression of Filaggrin and Bleomycin Hydrolase (BH) Genes (RNA Isolation and RT-PCR)

Neonatal keratinocytes (Lonza) were plated onto a 60-mm cell culture dish at a density of 1.25×10⁶ cells/dish using KGM medium (Lonza, human epidermal growth factor, insulin, BPE (bovine pituitary extract), hydrocortisone, epinephrine, transferrin and gentamicin/amphotericin B) and cultured in a 5% CO₂ incubator at 37° C. until ˜80% confluency. The cells were treated with the bamboo sap extract of Example 1, the betaine of Example 2 or the mixture of the bamboo sap and the betaine of Example 3 at concentrations of 100 ppm, 100 ppm, and 100 (50+50) ppm, respectively, and then cultured for 4 days.

After removing the medium, RNA was isolated by adding 1 mL of TRIzol reagent (Invitrogen) according to the manufacturer's instruction. RT-PCR (reverse transcription-polymerase chain reaction) was conducted after quantifying RNA at 260 nm using a UV detector (Hewlett Packard). For genetic analysis for each sample, the result was normalized to the RPL13A gene.

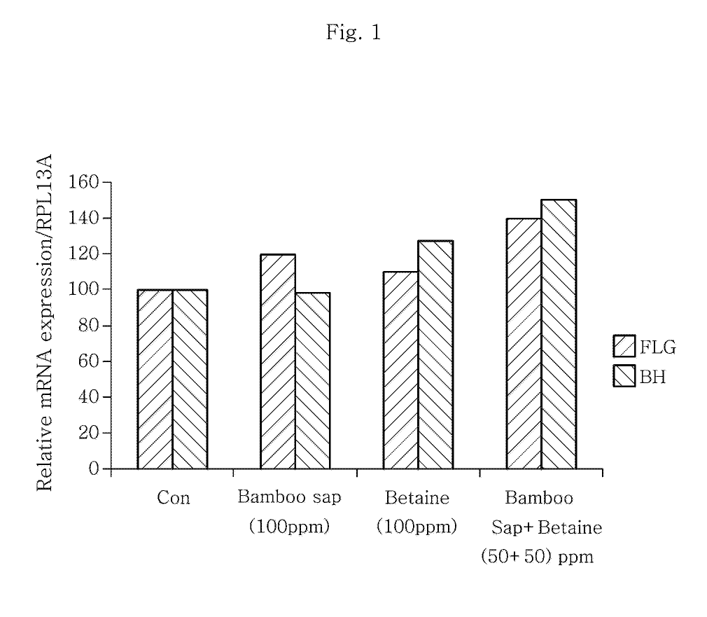

As a result, treatment of the keratinocytes with the mixture of the 50 ppm bamboo sap and the 50 ppm betaine (total 100 ppm) for 4 days resulted in about 20-30% increased expression of the filaggrin and bleomycin hydrolase genes as compared to when the cells were treated with the bamboo sap or the betaine alone. The result is shown in FIG. 1.

### TEST EXAMPLE 2

### Quantification of Natural Moisturizing Factors (NMFs)

Neonatal keratinocytes were plated onto a 60-mm cell culture dish at a density of 1.25×10⁶ cells/dish using KGM medium and cultured in a 5% CO₂ incubator at 37° C. until ˜80% confluency. The cells were treated with the bamboo sap extract of Example 1, the betaine of Example 2 or the mixture of the bamboo sap and the betaine of Example 3 at concentrations of 100 ppm, 100 ppm, and 100 (50+50) ppm, respectively, and then cultured for 4 days. After removing the medium and lysing the cells using a cell lysis buffer, NMFs were quantified for each sample by HPLC.

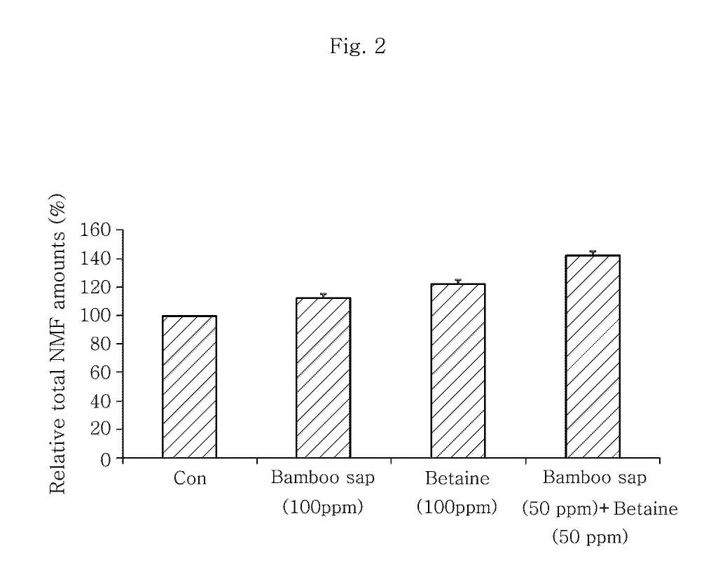

As a result, treatment of the cells with the mixture of the 50 ppm bamboo sap and the 50 ppm betaine (total 100 ppm) for 4 days resulted in increase of natural moisturizing factors by about 20-30% as compared to when the cells were treated with the bamboo sap or the betaine alone. FIG. 2 shows the result of comparing the total amount of natural moisturizing factors (total NMF) with that of the control group.

### TEST EXAMPLE 3

### Evaluation of Water Content in Skin

The skin moisturizing ability of the compositions of Comparative Example 1 and Examples 1-3 was evaluated as follows.

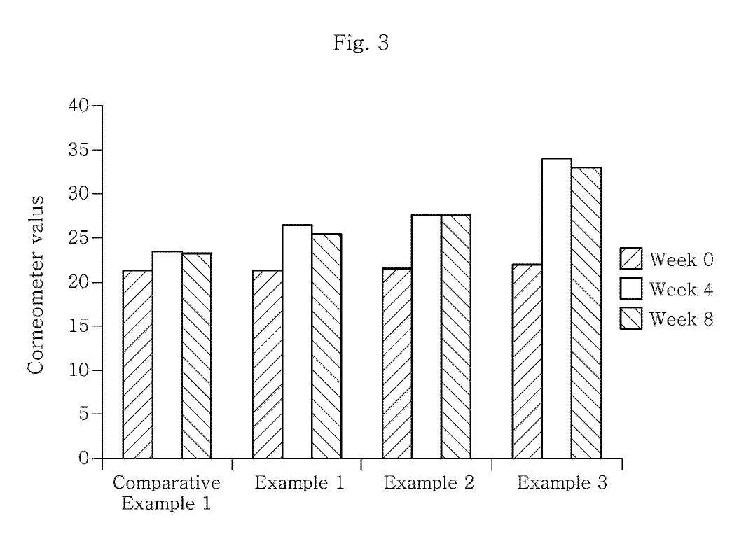

Forty (40) women in their 30s to 40s were divided into four groups with 10 people per group and were asked to apply the compositions of Comparative Example 1 and Examples 1-3 twice a day in the morning and evening for 8 weeks. Then, the water content in skin was measured using Corneometer (Germany). The result is shown in FIG. 3.

As a result, when the bamboo sap or the betaine were used alone (Examples 1-2), the water content in skin was increased by only 8-13% as compared to the control group which was not treated with the active ingredient. In contrast, when the mixture of the bamboo sap and the betaine was used (Example 3), a remarkably superior effect was observed as compared to the control group or Examples 1-2, because the water content in skin was increased by as much as 47% as compared to the control group which was not treated with the active ingredient. Accordingly, it was confirmed that a composition containing the mixture of the bamboo sap and the betaine as an active ingredient is suitable for use as a cosmetic composition for moisturizing skin or as a pharmaceutical composition for treating a skin dryness disease.

Hereinafter, the present disclosure will be described in detail through formulation examples. However, the scope of the present disclosure is not limited by them.

### FORMULATION EXAMPLE 1

### Preparation of Soft Capsule

A soft capsule filling solution was prepared by mixing 80 mg of a mixture of bamboo sap and betaine, 9 mg of vitamin E, 9 mg of vitamin C, 2 mg of palm oil, 8 mg of hydrogenated vegetable oil, 4 mg of yellow beeswax and 9 mg of lecithin according to a commonly employed method. Then, a soft capsule was prepared by filling 400 mg of the solution per capsule. Separately from this, a soft capsule sheet was prepared using 66 parts by weight of gelatin, 24 parts by weight of glycerin and 10 parts by weight of sorbitol and a soft capsule containing 400 mg of the composition according to the present disclosure was prepared by filling the filling solution therein.

### FORMULATION EXAMLE 2

### Preparation of Tablet

After mixing 80 mg of a mixture of bamboo sap and betaine with 9 mg of vitamin E, 9 mg of vitamin C, 200 mg of galactooligosaccharide, 60 mg of lactose and 140 mg of maltose and granulating using a fluidized-bed drier, 6 mg of sugar ester was added. A tablet was prepared from 504 mg of the resulting composition according to a commonly employed method.

### FORMULATION EXAMPLE 3

### Preparation of Drink

After mixing 80 mg of a mixture of bamboo sap and betaine with 9 mg of vitamin E, 9 mg of vitamin C, glucose 10 g, 0.6 g of citric acid and 25 g of oligosaccharide syrup and adding 300 mL of purified water, 200 mL of the resulting mixture was filled per bottle. Then, a drink was prepared by sterilizing at 130 ° C. for 4-5 seconds.

### FORMULATION EXAMPLE 4

### Preparation of Granule

80 mg of a mixture of bamboo sap and betaine was mixed with 9 mg of vitamin E, 9 mg of vitamin C, 250 mg of microcrystalline glucose and 550 mg of starch. The resulting mixture was prepared into a granule using a fluidized-bed granulator and filled in a pouch.

### FORMULATION EXAMPLE 5

### Preparation of Softening Lotion (Skin Lotion)

A softening lotion was prepared according to a commonly employed softening lotion preparation method by uniformly mixing 255 mg of purified water, 0.3 mg of a mixture of bamboo sap and betaine, 6.0 mg of butylene glycol, 6.0 mg of propylene glycol, 0.3 mg of carboxyvinyl polymer, 0.6 mg of PEG-12 nonyl phenyl ether, 1.2 mg of polysorbate 80, 30.0 mg of ethanol, 0.3 mg of triethanolamine and small amounts of an antiseptic, a pigment and a fragrance.

### FORMULATION EXAMPLE 6

### Preparation of Nourishing Lotion (Milk Lotion)

A nourishing lotion was prepared according to a commonly employed nourishing lotion preparation method by uniformly mixing 234 mg of purified water, 0.3 mg of a mixture of bamboo sap and betaine, 12.0 mg of beeswax, 4.5 mg of polysorbate 60, 4.5 mg of sorbitan sesquioleate, 1.5 mg of liquid paraffin, 15 mg of Montana 202 (SEPPIC), 9.0 mg of glycerin, 9.0 mg of butylene glycol, 9.0 mg of propylene glycol, 0.3 mg of carboxyvinyl polymer, 0.6 mg of triethanolamine and small amounts of an antiseptic, a pigment and a fragrance.

### FORMULATION EXAMPLE 7

### Preparation of Massage Cream

A massage cream was prepared according to a commonly employed massage cream preparation method by uniformly mixing 70 mg of purified water, 0.3 mg of a mixture of bamboo sap and betaine, 30.0 mg of beeswax, 4.5 mg of polysorbate 60, 6.0 mg of PEG 60 hydrogenated castor oil, 2.4 mg of sorbitan sesquioleate, 120.0 mg of liquid paraffin, 15.0 mg of squalane, 12.0 mg of Montana 202 (SEPPIC), 15.0 mg of glycerin, 12.0 mg of butylene glycol, 12.0 mg of propylene glycol, 0.6 mg of triethanolamine and small amounts of an antiseptic, a pigment and a fragrance.

### FORMULATION EXAMPLE 8

### Preparation of Pack

A pack was prepared according to a commonly employed pack preparation method by uniformly mixing 0.5 mg of a mixture of bamboo sap and betaine, 1.0 mg of β-1,3-glucan, 13.0 mg of polyvinyl alcohol, 0.2 mg of sodium carboxymethyl cellulose, 5.0 mg of glycerin, 0.1 mg of allantoin, 6.0 mg of ethanol, 0.3 mg of PEG-12 nonyl phenyl ether, 0.3 mg of polysorbate 60 and small amounts of an antiseptic, a pigment and a fragrance and then adding purified water.

### FORMULATION EXAMPLE 9

### Preparation of Gel

A gel was prepared according to a commonly employed gel preparation method by uniformly mixing 0.05 mg of a mixture of bamboo sap and betaine, 0.1 mg of β-1,3-glucan, 0.05 mg of sodium ethylenediaminetetraacetate, 5.0 mg of glycerin, 0.3 mg of carboxyvinyl polymer, 5.0 mg of ethanol, 0.5 mg of PEG-60 hydrogenated castor oil, 0.3 mg of triethanolamine and small amounts of an antiseptic, a pigment and a fragrance and then adding purified water.

### FORMULATION EXAMPLE 10

### Preparation of Ointment as Formulation for External Application to Skin

An ointment was prepared according to a commonly employed ointment preparation method by uniformly mixing 2.0 mg of a mixture of bamboo sap and betaine, 10.0 mg of β-1,3-glucan, 10.0 mg of beeswax, 5.0 mg of polysorbate 60, 2.0 mg of PEG-60 hydrogenated castor oil, 0.5 mg of sorbitan sesquioleate, 5.0 mg of vaseline, 10.0 mg of liquid paraffin, 5.0 mg of squalane, 3.0 mg of shea butter, 5.0 mg of caprylic/capric triglyceride, 10 mg of glycerin, 10.2 mg of propylene glycol, 0.2 mg of triethanolamine and small amounts of an antiseptic, a pigment and a fragrance and then adding purified water.

### FORMULATION EXAMPLE 11

### Preparation of Medication for Topical Application (Patch)

A patch was prepared according to a commonly employed patch preparation method by uniformly mixing 1.0 mg of a mixture of bamboo sap and betaine, 3.0 mg of β-1,3-glucan, 20.0 mg of hexylene glycol, 0.7 mg of dimethylamine, 1.0 mg of polyacrylic acid (Carbopol 934P), 0.1 mg of sodium sulfite, 1.0 mg of polyoxyethylene lauryl ether (E.O=9), 1.0 mg of polyhydroxyethylene cetyl stearyl ether (Cetomacrogol 1000), 2.5 mg of viscous paraffin oil, 2.5 mg of caprylic acid ester/capric acid ester (Cetiol LC) and 3.0 mg of polyethylene glycol 400 and then adding purified water.

It is to be understood that those of ordinary skill in the art to which the present disclosure belongs can make various changes and modifications within the scope of the present disclosure.

What is claimed is:

1. A method for moisturizing a skin of a subject, which comprises administering an effective amount of a bamboo sap and betaine to a subject in need thereof.

2. The method according to claim 1, wherein the skin moisturizing is achieved through regulation of natural moisturizing factors (NMFs).

3. The method according to claim 2, wherein the bamboo sap and the betaine regulate natural moisturizing factors by increasing the expression of the filaggrin gene, increasing the expression of the bleomycin hydrolase gene or both.

4. The method according to claim 1, wherein the bamboo sap is the sap of one or more of Phyllostachys bambusoides, Phyllostachys nigra var. henonis and Phyllostachys pubescens.

5. The method according to claim 1, wherein the betaine is glycine betaine.

6. The method according to claim 1, wherein a weight ratio of the bamboo sap and the betaine as active ingredients is 1:0.1 to 1:10.

7. The method according to claim 1, wherein the bamboo sap is administered in the form of a composition and the bamboo sap is contained in an amount of 0.001-80 wt % based on the total weight of the composition.

8. The method according to claim 1, wherein the betaine is administered in the form of a composition and the betaine is contained in an amount of 0.001-80 wt % based on the total weight of the composition.

9. The method according to claim 1, wherein the bamboo sap and the betaine are administered in the form of a composition and the composition is a cosmetic composition.

10. The method according to claim 1, wherein the bamboo sap and the betaine are administered in the form of a composition and the composition is a pharmaceutical composition.

11. The method according to claim 1, wherein the bamboo sap and the betaine are administered in the form of a composition and the composition is a health food composition.

12. The method according to claim 10, wherein the pharmaceutical composition is a composition which is used to treat or prevent one or more skin dryness disease selected from atopy, xeroderma, xerotic eczema, psoriasis and xeroderma pigmentosum.

13. A kit for moisturizing skin, which comprises: a first composition comprising a bamboo sap; and a second composition comprising betaine.

14. The kit for moisturizing skin according to claim 13, wherein the concentration of the bamboo sap is 0.001-80 wt % based on the total weight of the first composition and the concentration of the betaine is 0.001-80 wt % based on the total weight of the second composition.

15. The kit for moisturizing skin according to claim 13, wherein the kit further comprises an instruction.

16. The kit for moisturizing skin according to claim 15, wherein the instruction dictates that the first composition and the second composition are administered at a weight ratio of 1:0.1-1:10
